(19) United States
(12) Patent Application Publication
(10) Pub. No.: US 2025/0329488 A1
(43) Pub. Date: Oct. 23, 2025

(54) A PLANAR TRANSFORMER ASSEMBLY, AN IMPLANTABLE MEDICAL DEVICE COMPRISING A PLANAR TRANSFORMER ASSEMBLY, AND A METHOD FOR FABRICATING A PLANAR TRANSFORMER ASSEMBLY

(71) Applicant: BIOTRONIK SE & Co. KG, Berlin (DE)

(72) Inventor: Colby WHITAKER, Oregon City, OR (US)

(73) Assignee: BIOTRONIK SE & Co. KG, Berlin (DE)

(21) Appl. No.: 18/862,589

(22) PCT Filed: Mar. 28, 2023
(86) PCT No.: PCT/EP2023/057912

§ 371 Date: Nov. 4, 2024

Related U.S. Application Data
(60) Provisional application No. 63/343,289, filed on May 18, 2022.

(30) Foreign Application Priority Data
Jun. 14, 2022 (EP) 22178906.8

(51) Int. Cl. H01F 27/28; A61N 1/375; H01F 38/00; H01F 41/04

(52) U.S. Cl. CPC H01F 27/2804; A61N 1/3758; H01F 38/00; H01F 41/041; H01F 2027/2809; H01F 2027/2819

(57) ABSTRACT

A planar transformer assembly includes a primary coil, a multiplicity of secondary coils magnetically coupled to the primary coil, at least one printed circuit board, a multiplicity of layers formed on the at least one printed circuit board, and a multiplicity of groups of coil windings forming said primary coil and said multiplicity of secondary coils. Each group includes a first primary coil winding of said primary coil arranged on a first layer of said multiplicity of layers and a second primary coil winding of said primary coil arranged on a second layer of said multiplicity of layers, and a multiplicity of secondary coil windings of said multiplicity of secondary coils arranged on at least one intermediate layer of said multiplicity of layers in between said first layer and said second layer.

### CROSS-REFERENCE TO RELATED APPLICATIONS

This application is the United States National Phase under 35 U.S.C. § 371 of PCT International Patent Application No. PCT/EP2023/057912, filed on Mar. 28, 2023, which claims the benefit of European Patent Application No. 22178906.8, filed on Jun. 14, 2022 and U.S. Provisional Patent Application No. 63/343,289, filed on May 18, 2022, the disclosures of which are hereby incorporated by reference herein in their entireties.

### TECHNICAL FIELD

The instant invention relates to a planar transformer assembly, to an implantable medical device comprising a planar transformer assembly, and to a method for fabricating a planar transformer assembly.

### BACKGROUND

Within a planar transformer assembly, a primary coil and a secondary coil are formed by windings formed on one or multiple printed circuit boards forming a multiplicity of layers on which the windings are arranged. The layers herein are stacked along a longitudinal direction, the windings extending circumferentially about the longitudinal direction. The secondary coil herein is magnetically coupled to the primary coil by means of a magnetic core about which the windings extend.

Implantable medical devices, for example, stimulation devices such as defibrillator devices (e.g., so-called implantable cardioverter defibrillators, in short ICD) typically are small in size to allow implantation into a patient. A transformer assembly to be included in an implanted medical device hence needs to have a space-efficient construction such that the transformer assembly may be included in a housing of the implantable medical device. As a planar transformer assembly using one or multiple stacked printed circuit boards with windings formed on the printed circuit boards by means of printed circuit board techniques may be constructed to be small in size while nevertheless using windings with a comparatively large number of turns, typically a planar transformer assembly is used in an implantable medical device, for example, in a defibrillator device, for example, to charge capacitors for providing energy to electrodes for performing a stimulation action, such as a defibrillation action.

U.S. Pat. No. 9,579,517 describes an implantable medical device in the shape of an implantable cardioverter defibrillator (ICD) comprising a transformer assembly having two primary coils and a multiplicity of secondary coils for charging capacitors. The secondary coils are magnetically coupled to the primary coils by means of a magnetic core, wherein the secondary coils are wound about the core and each are associated with a capacitor to deliver charge to the associated capacitor.

U.S. Publication No. 2014/0368308 A1 describes a planar transformer assembly for use in charging capacitors of an implantable cardioverter defibrillator (ICD), the planar transformer assembly including windings arranged to minimize voltage across intervening dielectric layers. Each secondary winding of a plurality of secondary windings herein is arranged relative to a primary winding, in a hierarchical fashion, such that a DC voltage, with respect to ground, of a first secondary winding is lower than that of a second secondary winding. The first secondary winding herein is in closest proximity to the primary winding. The primary winding and the secondary windings are formed on a corresponding plurality of dielectric layers.

When multiple secondary coils are used in a planar transformer assembly, it is desirous to form the secondary coils such that they exhibit matching inductances. If inductances of the secondary coils are not matched, the use of a planar transformer assembly may be limited to applications requiring a rather low precision. In particular for a use in implantable medical devices such as cardioverter defibrillators (ICDs) it may be beneficial to obtain a good matching in between secondary coils of a planar transformer assembly.

The present disclosure is directed toward overcoming one or more of the above-mentioned problems, though not necessarily limited to embodiments that do.

### SUMMARY

It is an object of the instant invention to provide a planar transformer assembly, an implantable medical device and a method for fabricating a planar performer assembly which allow to fabricate the transformer assembly with multiple secondary coils while achieving a good matching performance between the secondary coils.

In one aspect, a planar transformer assembly comprises: a primary coil, a multiplicity of secondary coils magnetically coupled to the primary coil, at least one printed circuit board, a multiplicity of layers formed on the at least one printed circuit board, and a multiplicity of groups of coil windings forming said primary coil and said multiplicity of secondary coils. Each group comprises a first primary coil winding of said primary coil arranged on a first layer of said multiplicity of layers and a second primary coil winding of said primary coil arranged on a second layer of said multiplicity of layers, and a multiplicity of secondary coil windings of said multiplicity of secondary coils arranged on at least one intermediate layer of said multiplicity of layers in between said first layer and said second layer.

By forming multiple groups of coil windings, the coil windings of the primary coil and the multiplicity of secondary coils are distributed across the stack of layers on which the windings are formed. The layers herein generally are stacked along a longitudinal direction, wherein the layers may be formed by a single, multilayered printed circuit board or by multiple stacked printed circuit boards, each of which may comprise one or multiple layers of conductor material embedded in dielectric material for forming the windings.

In one embodiment, each group of the multiplicity of groups is formed on a dedicated printed circuit board. Hence, the planar transformer assembly comprises multiple printed circuit boards, on each of which one group of the multiplicity of groups of coil windings is formed.

Each group of coil windings comprises a first primary coil winding and a second primary coil winding. The first primary coil winding is arranged on a first layer, and the second primary coil winding is arranged on a second layer. In between the first layer and the second layer multiple secondary coil windings forming the multiple secondary coils are arranged. Hence, on intermediate layers in between the first layer and the second layer, which each carry a primary coil winding, the secondary coil windings are formed, such that the secondary coil windings are arranged in between an arrangement of primary coil windings.

The primary coil windings together form the primary coil. The secondary coil windings in turn form the multiple secondary coils. Each winding herein may comprise one or multiple turns, formed by etched conduction paths on a corresponding layer. A turn ratio in between the primary coil and each of the secondary coils may be set such that a desired transformation ratio is obtained.

The first layer herein may be an outer layer of the corresponding group. Likewise, the second layer may be an outer layer of the corresponding group opposite the first layer. In another embodiment, however, further layers are arranged outside of the first layer and/or the second layer carrying further first or secondary coil windings.

As the transformer assembly comprises multiple groups, multiple arrangements of primary coil windings with secondary coil windings formed therebetween are employed. The different groups herein may have a similar order of coil windings, or may differ in the order of their coil windings.

Because the windings are distributed across the length of the planar transformer assembly (measured along the longitudinal direction along which the layers of the planar transformer assembly are stacked), the inductances of the secondary coils may be matched such that the secondary coils exhibit substantially equivalent inductances. In particular, it may be achieved that the secondary windings of each secondary coil are exposed to an area of a magnetic core coupling the primary coil to the secondary coils which is similar to the area of exposure of the other secondary coils. In addition, the secondary coil windings of each secondary coil may have a distribution with respect to the primary coil windings which is electrically similar to the other secondary coils.

In one embodiment, the multiplicity of secondary coil windings includes a first secondary coil winding of a first secondary coil, a second secondary coil winding of a second secondary coil and a third secondary coil winding of a third secondary coil. The planar transformer assembly hence comprises (at least) three secondary coils, wherein each group of coil winding comprises windings of the first secondary coil, the second secondary coil and the third secondary coil. Each coil winding of the first secondary coil, the second secondary coil and the third secondary coil may comprise one or multiple turns wound about the longitudinal axis, in particular about a magnetic core extending across the layers in order to establish a magnetic coupling in between the primary coil and the secondary coils.

In one embodiment, the first secondary coil winding, the second secondary coil winding and the third secondary coil winding are arranged on different intermediate layers in between the first layer and the second layer, on which primary coil windings are arranged. The first secondary coil winding, the second secondary coil winding and the third secondary coil winding hence are displaced with respect to one another on intermediate layers in between the layers of the primary coil windings. In particular, the secondary coil windings of the first secondary coil, the second secondary coil and the third secondary coil may be arranged to alternate, a coil winding of one secondary coil in each case being adjacent to a coil winding of another secondary coil.

In one embodiment, in addition or alternatively to arranging the coil windings of the secondary coils on different intermediate layers, a first secondary coil winding, a second secondary coil winding and a third secondary coil winding may be arranged on a common intermediate layer. A layer hence may comprise windings of different secondary coils. The windings of the secondary coils hence do not (only) alternate along the longitudinal direction along which the layers are stacked, but also within the layers.

In one embodiment, the multiplicity of secondary coil windings includes at least one of multiple first secondary coil windings of the first secondary coil, multiple second secondary coil windings of the secondary coil and multiple third secondary coil windings of the third secondary coil. In each group, hence, multiple first secondary coil windings, multiple second secondary coil windings and/or multiple third secondary coil windings may be present. In one embodiment, each group comprises multiple first secondary coil windings, multiple second secondary coil windings and multiple third secondary coil windings. Within each group multiple coil windings of multiple secondary coils hence are arranged in between the primary coil windings on the first layer and the second layer.

In one embodiment, the first secondary coil windings, the second secondary coil windings and the third secondary coil windings are arranged on different intermediate layers. The first secondary coil windings, the second secondary coil windings and the third secondary coil windings herein may alternate such that a secondary coil winding of one secondary coil on one intermediate layer is in each case adjacent to a secondary coil winding of another secondary coil on another intermediate layer.

The first secondary coil windings, the second secondary coil windings, and the third secondary coil windings, in one embodiment, are respectively connected to each other. In particular, the first secondary coil windings may be electrically connected by first vias, the second secondary coil windings may be electrically connected by second vias and the third secondary coil windings may be electrically connected by third vias. By means of the vias, electrical interconnections in between different layers of the stack of layers of the planar transformer assembly are established, such that coil windings on different layers of the planar transformer assembly are electrically interconnected.

In one embodiment, a first secondary coil winding is arranged on a first intermediate layer, and a second secondary coil winding is arranged on a second intermediate layer adjacent to the first intermediate layer. Herein, in one embodiment, in addition a third secondary coil winding is arranged on a third intermediate layer adjacent to the second intermediate layer. Hence, a stack of secondary coil windings is formed in which a first secondary coil winding, a second secondary coil winding and a third secondary coil winding are stacked upon one another. Such sequence may be repeated within each group, wherein it also is possible to form different sequences, such that, for example, a first secondary coil winding is followed by a second secondary coil winding, which again is followed by a first secondary coil winding, or the like.

Generally, the secondary coil windings may be arranged in between the primary coil windings such that the secondary coil windings of the different secondary coils alternate across the different intermediate layers.

In one embodiment, at least some intermediate layers may each include at least two of a first secondary coil winding, a second secondary coil winding and a third secondary coil winding. On an intermediate layer, hence, multiple coil windings of different secondary coils are formed, for example, a first secondary coil winding, a secondary coil winding and a third secondary coil winding.

In one embodiment, the transformer assembly comprises a first group of coil windings at a first end of the transformer assembly and a second group of coil windings at a second end of the transformer assembly. Herein, the first group comprises a stack of at least a first secondary coil winding, a second secondary coil winding and a third secondary coil winding of a first order. For example, when viewed in a direction pointing from the first group towards the second group, within the first group an order of windings may be formed in which a first secondary coil winding is followed by a second secondary coil winding, which again is followed by a third secondary coil winding. In contrast, the second group may comprise a stack of at least a first secondary coil winding, a second secondary coil winding and a third secondary coil winding of a second order inverse to the first order, when viewed in a direction pointing from the second group towards the first group. The order of the second group hence may be inverse with respect to the order of the first group. In the second group, hence, for example, when viewed in a direction from the second group towards the first group, a third secondary coil winding of a third secondary coil may be followed by a second secondary coil winding of a second secondary coil, which again is followed by a first secondary coil winding of a first secondary coils.

In one embodiment, the stack of the first group is arranged in proximity to an outermost arrangement of primary coil windings of the first group. Hence, the stack forms an outermost sequence of the secondary coil windings of the first group, when viewed in a direction pointing from the first group towards the second group (along the longitudinal direction along which the layers of the planar transformer assembly are stacked). In contrast, the stack of the second group may be arranged in proximity to an outermost arrangement of primary coil windings of the second group, such that the stack forms an outermost sequence of the secondary coil windings of the second group, when viewed in a direction pointing from the second group towards the first group.

By having an inverse order in the first group in comparison to the second group, it may be achieved that all secondary coils are exposed to an equivalent magnetic core area of a magnetic core magnetically coupling the primary coil to the secondary coils.

In another aspect, an implantable medical device comprises a planar transformer assembly of the kind described above. An implantable medical device of this kind may, for example, be implantable into a patient's heart. The implantable medical device may, for example, be a stimulation device, such as a pacemaker device or a defibrillator device, in particular in the shape of an implantable cardioverter defibrillator (ICD). The planar transformer assembly herein may serve to charge capacitors for outputting stimulation energy towards an electrode arrangement for emitting stimulating signals.

In yet another aspect, a method for fabricating a planar transformer assembly comprises: forming a multiplicity of layers on at least one printed circuit board to form a primary coil and a multiplicity of secondary coils to be magnetically coupled to the primary coil, wherein said forming includes: forming a multiplicity of groups of coil windings to form said primary coil and said multiplicity of secondary coils, wherein for each group a first primary coil winding of said primary coil is arranged on a first layer of said multiplicity of layers and a second primary coil winding of said primary coil is arranged on a second layer of said multiplicity of layers, and a multiplicity of secondary coil windings of said multiplicity of secondary coils is arranged on at least one intermediate layer of said multiplicity of layers in between said first layer and said second layer.

The advantages and advantageous embodiments described above for the transformer assembly equally apply also to the method, such that it shall be referred to the above in this respect.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The various features and advantages of the present invention may be more readily understood with reference to the following detailed description and the embodiments shown in the drawings. Herein,

FIG. 1 shows a schematic drawing of an implantable medical device in an implanted state within a patient;

FIG. 2 shows a schematic functional drawing of a transformer assembly of an implantable medical device;

FIG. 3 shows a schematic view of an embodiment of a planar (PCB) transformer assembly;

FIG. 4 shows a view of a single layer of the transformer assembly;

FIG. 5 shows a view of another embodiment of a planar (PCB) transformer assembly; and

FIG. 6 shows a view of a layer of a transformer assembly, according to one embodiment.

### DETAILED DESCRIPTION

Subsequently, embodiments of the present invention shall be described in detail with reference to the drawings. In the drawings, like reference numerals shall designate functionally similar structural elements, if appropriate.

It is to be noted that the embodiments are not limiting for the present invention, but merely represent illustrative examples.

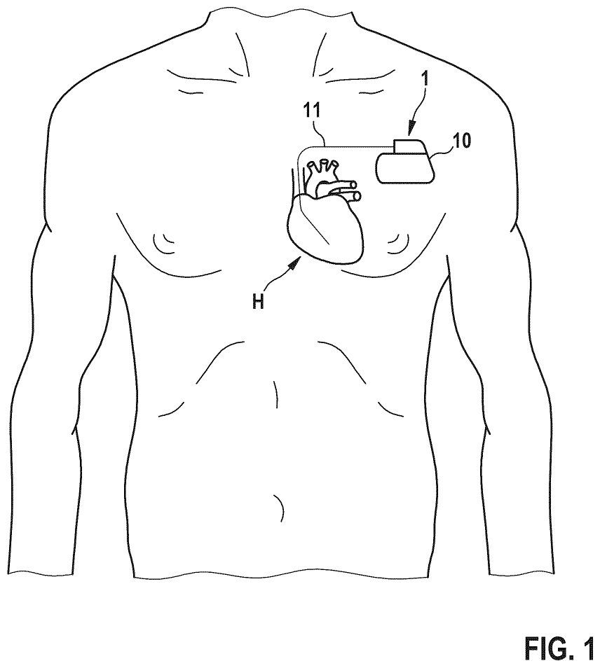

Referring now to FIG. 1, an implantable medical device 1 such as a stimulation device, for example, a defibrillator device such as an implantable cardioverter defibrillator (ICD), may be implanted in a patient in order to provide a stimulation action in a patient's heart H. The implantable medical device 1 herein may, for example, comprise a generator 10, which, for example, may be subcutaneously implanted in a patient, and one or multiple leads 11 carrying one or multiple electrodes extending from the generator 10 in order to inject an electrical stimulation signal into the patient's heart H and/or to receive sense signals.

Implantable medical devices 1, as schematically shown in FIG. 1, generally are small in size. Components used within an implantable medical device 1, such as a processor, an energy storage device in the shape of a battery, and electrical circuitry hence likewise need to be small in size for integration into a housing of the implantable medical device 1.

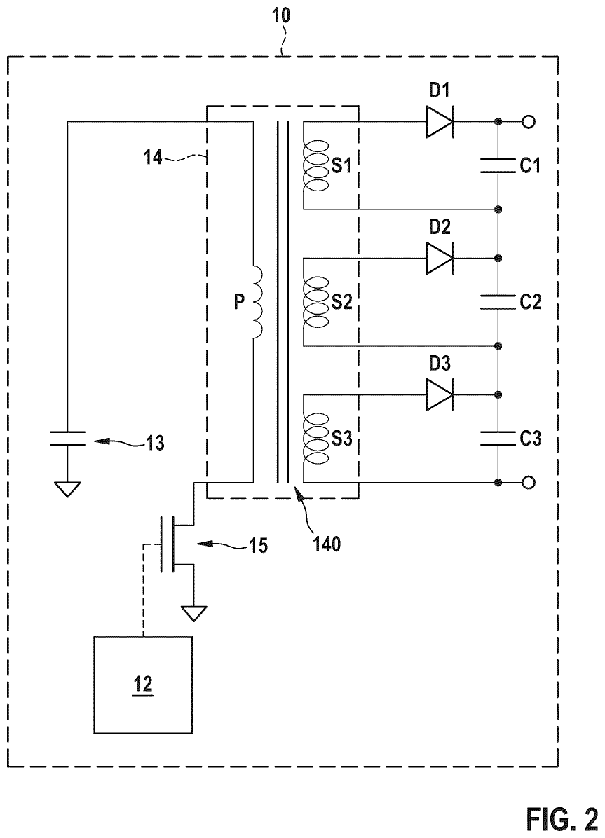

Referring now to FIG. 2, an implantable medical device 1 such as a defibrillator device may, for example, comprise a transformer assembly 14 which serves to transform energy of an energy storage 13 in the shape of a battery to charge capacitors C1, C2, C3 in order to inject, e.g., stimulation energy for the purpose of defibrillation into a patient. A primary coil P of the transformer assembly 14 herein is, for example, selectively connected to the battery 13 by means of a switch device 15, which is controlled by a controller 12. Within the transformer assembly 14, the primary coil P is magnetically coupled by means of a magnetic core 140 to a multiplicity of secondary coils S1, S2, S3, which, e.g., via an electrical circuitry including diodes D1, D2, D3 are connected to the arrangement of capacitors C1, C2, C3 for charging the capacitors C1, C2, C3 selectively from the battery 13.

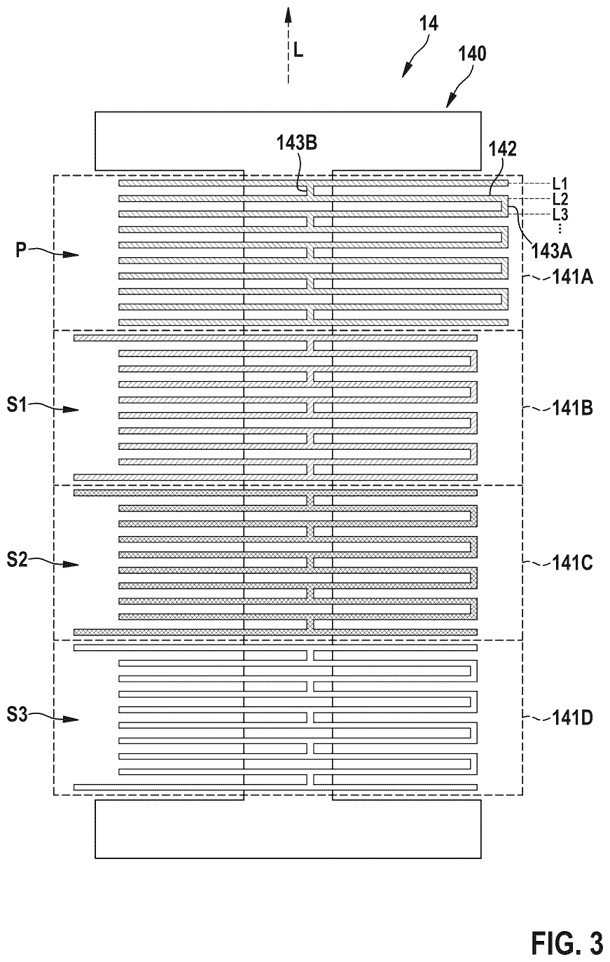

A transformer assembly 14 as it is schematically shown in FIG. 2 may conventionally be, for example, formed as a planar transformer assembly, as shown in an example in FIG. 3. The planar transformer assembly 14 comprises printed circuit boards 141A, 141B, 141C, 141D, each of which comprises layers L1 . . . L3 of conductive paths by means of which coil windings 142 are formed. A first printed circuit board 141A herein, with its coil windings 142 on the layers L1 . . . L3, may form the primary coil P, a second printed circuit board 141B may form a first secondary coil S1, a third printed circuit board 141C may form a second secondary coil S2, and a fourth printed circuit board 141D may form a third secondary coil S3. The primary coil P and the secondary coils S1, S2, S3 are magnetically coupled by means of a magnetic core 140 extending along a longitudinal direction L through the windings of the different coils P, S1, S2, S3. The windings of the coils P, S1, S2, S3 are arranged on the different layers L1 . . . L3 forming a stack along the longitudinal direction L. The coil windings 142 on the different layers L1 . . . L3 are electrically interconnected by means of vias 143A, 143B such that coils P, S1, S2, S3 are formed within the transformer assembly 14.

In a transformer assembly 14 as shown in FIG. 3, the windings 142 are formed by conductive paths within multilayered printed circuit boards 141A-141D, the windings on the different layers L1 . . . L3 being electrically isolated by means of dielectric material of the printed circuit boards 141A-141D in which the conductive paths are embedded.

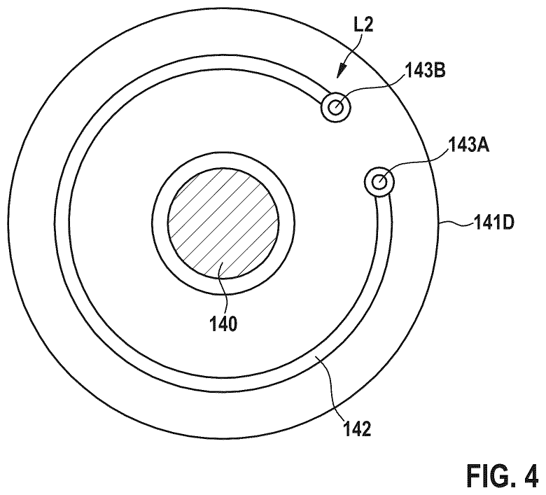

Each layer L1 . . . L3, as shown schematically in FIG. 4 for a layer L2, forms a coil winding 142 extending about the longitudinal direction L and about the magnetic core 140 reaching through the different layers L1 . . . L3. By electrically interconnecting the windings 142 of the different layers L1 . . . L3 in series by means of the vias 143A, 143B, the coils P, S1, S2, S3 are formed.

In the transformer assembly 14 of FIG. 3, the windings of the different coils P, S1, S2, S3 are grouped and spatially separated from one another. This may cause inductances of the secondary coils S1, S2, S3 to not match, potentially limiting the applicability of a transformer assembly 14 as shown in FIG. 3, in particular for applications which require a precise inductance matching of the secondary coils S1-S3.

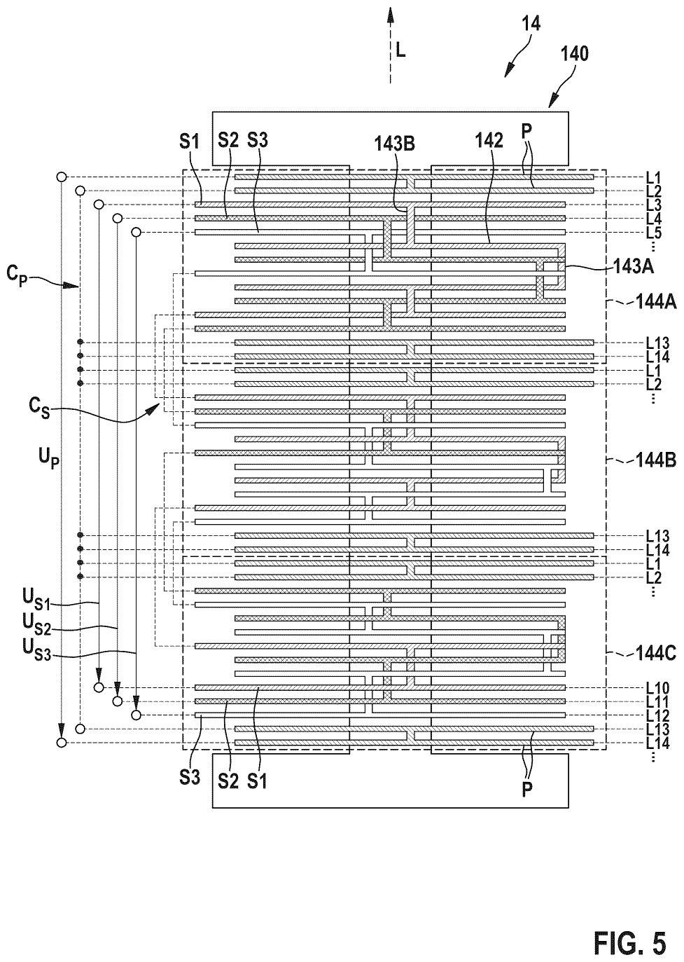

For this reason, it herein is proposed to form groups of coil windings 142 in which windings of different coils P, S1, S2, S3 are interleaved with one another, as this is shown in one embodiment in FIG. 5.

In the embodiment of FIG. 5, three groups 144A, 144B, 144C of coil windings are formed, wherein each group 144A, 144B, 144C may be implemented on a dedicated printed circuit board, such that in the embodiment of FIG. 5 three printed circuit boards are stacked on one another in order to form a primary coil P and three secondary coils S1, S2, S3.

It shall be noted that a transformer assembly 14 as described herein may comprise two or more secondary coils S1, S2, S3, for example, three secondary coils or more than three secondary coils.

Within the embodiment of FIG. 5, each group 144A, 144B, 144C comprises outer coil windings of the primary coil P, in between which secondary coil windings of the secondary coils S1, S2, S3 are arranged. In particular, in each group on the two outermost layers L1, L2, L13, L14 (of the 14 total layers L1 . . . L14 of the group 144A, 144B, 144C) primary coil windings are formed. On intermediate layers L3 . . . L12 in between the outer layers L1, L2, L13, L14, in contrast, secondary coil windings are arranged to form the secondary coils S1, S2, S3.

Herein, the secondary coil windings alternate, such that each secondary coil winding of one secondary coil S1, S2, S3 on one layer L3 . . . L12 is followed by a secondary coil winding of another secondary coil S1, S2, S3 on an adjacent layer L3 . . . L12.

Namely, the different groups 144A, 144B, 144C comprise sequences of coil windings as indicated in table 1 (group 144A), table 2 (group 144B) and table 3 (group 144C) below:

TABLE 1
Sequence of coil windings of the coils on the different
layers of group 144A according to FIG. 5

| Layer | Coil winding of |
|---|---|
| L1 | P |
| L2 | P |
| L3 | S1 |
| L4 | S2 |
| L5 | S3 |
| L6 | S1 |
| L7 | S2 |
| L8 | S3 |
| L9 | S1 |
| L10 | S2 |
| L11 | S1 |
| L12 | S2 |
| L13 | P |
| L14 | P |

TABLE 2
Sequence of coil windings of the coils on the different
layers of group 144B according to FIG. 5

| Layer | Coil |
|---|---|
| L1 | P |
| L2 | P |
| L3 | S1 |
| L4 | S2 |
| L5 | S3 |
| L6 | S1 |
| L7 | S2 |
| L8 | S3 |
| L9 | S1 |
| L10 | S3 |
| L11 | S1 |
| L12 | S3 |
| L13 | P |
| L14 | P |

TABLE 2
Sequence of coil windings of the coils on the different
layers of group 144C according to FIG. 5

| Layer | Coil |
|---|---|
| L1 | P |
| L2 | P |
| L3 | S2 |
| L4 | S3 |
| L5 | S2 |
| L6 | S3 |
| L7 | S1 |
| L8 | S2 |
| L9 | S3 |
| L10 | S1 |
| L11 | S2 |
| L12 | S3 |
| L13 | P |
| L14 | P |

Namely, in group 144A primary coil windings of the primary coil P are formed on the outermost layers L1, L2. On layer L3 a first secondary coil winding of the first secondary coil S1 is arranged, followed by a second secondary coil winding of the second secondary coil S2 on layer L4 and a third secondary coil winding of the third secondary coil S3 on the subsequent layer L5. In an alternating fashion further secondary coil windings of the secondary coils S1, S2, S3 are arranged on the further layers, wherein the group 144A is concluded by primary coil windings of the primary coil P on the two bottom layers L13, L14 of the group 144A.

Groups 144B and 144C comprise sequences of alternating secondary coil windings of the secondary coils S1, S2, S3 on intermediate layers in between respective outer pairs of layers L1, L2, L13, L14, on which primary coil windings of the primary coil P are arranged. The sequences of all groups 144A, 144B, 144C herein are not identical.

The sequences of the two outer groups 144A, 144C herein with respect to the respective outermost secondary coil windings are inversed.

Namely, group 144A on layers L3, L4, L5 comprises a first secondary coil winding of the first secondary coil S1 (layer L3), a second secondary coil winding of the second secondary coil S2 (layer L4) and a third secondary coil winding of the third secondary coil S3 (layer L5). When viewed in a direction pointing from group 144A towards group 144C, a sequence of a first secondary coil winding, a second secondary coil winding and a third secondary coil winding hence is formed.

In contrast, group 144C on layers L12, L11, L10 comprises a third secondary coil winding of the third secondary coil S3 (layer L12), a second secondary coil winding of the second secondary coil S2 (layer L11) and a first secondary coil winding of the first secondary coil S1 (layer L10). Hence, when viewed in a direction from group 144C towards group 144A an outermost sequence of secondary coil windings is obtained comprising, in this order, a third secondary coil winding, a second secondary coil winding and a first secondary coil winding, which is inverse to the outermost sequence of secondary coil windings of group 144A (when viewing the sequences in each case from the outside towards the inside of the stack).

In this way it is achieved that the secondary coils S1, S2, S3 each are substantially exposed to an equivalent area of the magnetic core 140, which helps to establish equivalent inductances of the secondary coils S1, S2, S3.

The coil windings on the different layers L1 . . . L14 of each group 144A, 144B, 144C are interconnected by vias 143A, 143B, such that the coil windings of the particular coils P, S1, S2, S3 are connected in series.

To connect the coil windings across the different groups 144A, 144B, 144C, which may be formed on different printed circuit boards, the coil windings are, for example, electrically interconnected by means of an interconnection system in the shape of conducting paths formed outside of the printed circuit boards, or by vias within the printed circuit boards. In particular, interconnections C_{P} serve to interconnect the primary coil windings of the primary coil P on the outer layers L1, L2, L13, L14 within a particular group 144A, 144B, 144C and across the groups 144A, 144B, 144C. Interconnections C_{S} serve to interconnect the secondary coil windings of the different secondary coils S1, S2, S3 across the different groups 144A, 144B, 144C.

At ports arranged on the two outer groups 144A, 144C voltage signals U_{P}, U_{S1}, U_{S2}, U_{S3} may be fed into respectively received from the primary coil P and the secondary coils S1, S2, S3.

In the embodiment of FIG. 5, on each intermediate layer L3 . . . L12 of each group 144A, 144B, 144C one coil winding of one secondary coil S1, S2, S3 may be formed, wherein the windings each may comprise one or multiple turns formed by etched conducting paths on the particular layers L3 . . . L12.

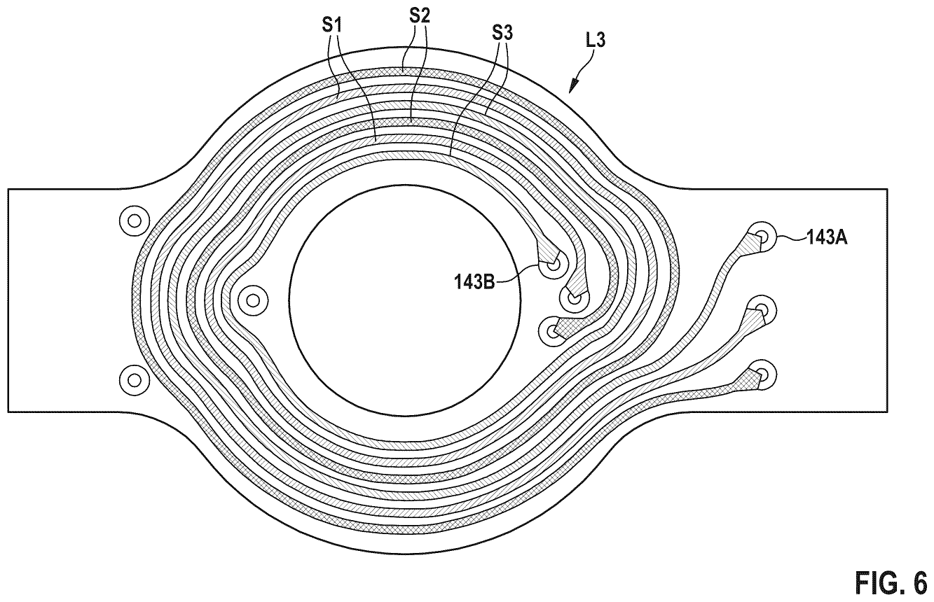

In another embodiment, as shown in FIG. 6, at least on some layers L3 . . . L12 multiple coil windings of the different secondary coils S1, S2, S3 may be arranged, such that a particular layer L3 . . . L12 carries windings of all or at least a subgroup of the secondary coils S1, S2, S3. As visible from FIG. 6, the coil windings of the different secondary coils S1, S2, S3 herein are arranged to alternate in a radial direction with respect to the longitudinal direction L, such that a coil winding of one secondary coil S1, S2, S3 is followed by a coil winding of another secondary coil S1, S2, S3 in the radial direction.

It is conceivable that all intermediate layers L3 . . . L12 carry coil windings of all secondary coils S1, S2, S3, as shown in FIG. 6, wherein the alternating sequence of coil windings may vary across the different layers L3 . . . L12. It however likewise is conceivable that only a subgroup of the intermediate layers L3 . . . L12 carries coil windings of all secondary coils S1, S2, S3.

By interleaving the coil windings of the secondary coils S1, S2, S3 and by arranging the coil windings of the secondary coils S1, S2, S3 in between the coil windings of the primary coil P in a grouped fashion, it may be achieved that the inductances of the secondary coils S1, S2, S3 are matched. In measurements it has been found that a mismatch in between the inductances of the secondary coils in an arrangement as shown in FIG. 5 may be reduced to a value below 0.5%, for example, below 0.25%.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### List of Reference Numerals

1 Implantable medical device
10 Generator
11 Electrode lead
12 Controller
13 Energy storage (battery)
14 Transformer assembly
140 Magnetic core
141A-141D Printed circuit board
142 Layer winding
143A, 143B Connection (via)
144A-144D Printed circuit board
15 Switch device
C1, C2, C3 Capacitor
C_{P}, C_{S} Connections
D1, D2, D3 Diode
H Heart
L Longitudinal axis
L1 . . . L14 Layer
P Primary coil
S1, S2, S3 Secondary coil
U_{P} Primary coil voltage
U_{S1}, U_{S2}, U_{S3} Secondary coil voltage

1. A planar transformer assembly, comprising:
a primary coil,
a multiplicity of secondary coils magnetically coupled to the primary coil,
at least one printed circuit board,
a multiplicity of layers (L1 . . . L14) formed on the at least one printed circuit board, and
a multiplicity of groups of coil windings forming said primary coil and said multiplicity of secondary coils, each group comprising
a first primary coil winding of said primary coil arranged on a first layer of said multiplicity of layers and a second primary coil winding of said primary coil arranged on a second layer of said multiplicity of layers, and
a multiplicity of secondary coil windings of said multiplicity of secondary coils arranged on at least one intermediate layer of said multiplicity of layers in between said first layer and said second layer.

2. The planar transformer assembly according to claim 1, comprising multiple printed circuit boards, wherein each of said multiplicity of groups is formed on one printed circuit board of said multiple printed circuit boards.

3. The planar transformer assembly according to claim 1, wherein said multiplicity of secondary coil windings includes a first secondary coil winding of a first secondary coil, a second secondary coil winding of a second secondary coil and a third secondary coil winding of a third secondary coil.

4. The planar transformer assembly according to claim 3, wherein said first secondary coil winding, said second secondary coil winding and said third secondary coil winding are arranged on different intermediate layers in between said first layer and said second layer.

5. The planar transformer assembly according to claim 3, wherein said first secondary coil winding, said second secondary coil winding and said third secondary coil winding are arranged on a common intermediate layer in between said first layer and said second layer.

6. The planar transformer assembly according to claim 1, wherein said multiplicity of secondary coil windings includes at least one of multiple first secondary coil windings of the first secondary coil, multiple second secondary coil windings of the second secondary coil and multiple third secondary coil windings of the third secondary coil.

7. The planar transformer assembly according to claim 6, wherein said first secondary coil windings, said second secondary coil windings and said third secondary coil windings are arranged on different intermediate layers.

8. The planar transformer assembly according to claim 7, wherein the first secondary coil windings are electrically connected by first vias, the second secondary coil windings are electrically connected by second vias, and the third secondary coil windings are electrically connected by third vias.

9. The planar transformer assembly according to claim 7, wherein a first secondary coil winding is arranged on a first intermediate layer, and a second secondary coil winding is arranged on a second intermediate layer adjacent to said first intermediate layer.

10. The planar transformer assembly according to claim 9, wherein a third secondary coil winding is arranged on a third intermediate layer adjacent to said second intermediate layer.

11. The planar transformer assembly according to claim 7, wherein at least some intermediate layers each include at least two of a first secondary coil winding, a second secondary coil winding and a third secondary coil winding.

12. The planar transformer assembly according to claim 1, wherein the transformer assembly comprises a first group of coil windings at a first end of the transformer assembly and a second group of coil windings at a second end of the transformer assembly, wherein the first group comprises a stack of at least a first secondary coil winding, a second secondary coil winding, and a third secondary coil winding of a first order, when viewed in a direction pointing from the first group towards the second group, and the second group comprises a stack of at least a first secondary coil winding, a second secondary coil winding, and a third secondary coil winding of a second order inverse to the first order, when viewed in a direction pointing from the second group towards the first group.

13. The planar transformer assembly according to claim 12, wherein the stack of the first group is arranged in proximity to an outermost arrangement of primary coil windings of the first group, and the stack of the second group is arranged in proximity to an outermost arrangement of primary coil windings of the second group.

14. An implantable medical device comprising a planar transformer assembly of claim 1.

15. A method for fabricating a planar transformer assembly, comprising:
forming a multiplicity of layers on at least one printed circuit board to form a primary coil and a multiplicity of secondary coils to be magnetically coupled to the primary coil,
wherein said forming includes: forming a multiplicity of groups of coil windings to form said primary coil and said multiplicity of secondary coils, wherein for each group
a first primary coil winding of said primary coil is arranged on a first layer of said multiplicity of layers and a second primary coil winding of said primary coil is arranged on a second layer of said multiplicity of layers, and
a multiplicity of secondary coil windings of said multiplicity of secondary coils is arranged on at least one intermediate layer of said multiplicity of layers in between said first layer and said second layer.
